(19) United States
(12) Patent Application Publication
(10) Pub. No.: US 2004/0230057 A1
(43) Pub. Date: Nov. 18, 2004

(54) Dye for optical recording medium

(76) Inventors: Chao-Nan Kuo, Hsinchu (TW); Sung-Kuei Chiang, Tzauchiau Shiang (TW)

Correspondence Address: J.C. Patents, Inc., Suite 250, 4 Venture, Irvine, CA, 92618, US

(21) Appl. No.: 10/820,600

(22) Filed: Apr. 7, 2004

(30) Foreign Application Priority Data
May 14, 2003 (TW) 92113053

(51) Int. Cl. C07D 209/56; G11B 7/24; C07D 453/02

(52) U.S. Cl. 546/134; 430/270.18; 546/135; 548/427; 548/469

(57) ABSTRACT

An optical recording medium dye has the following structure (1):

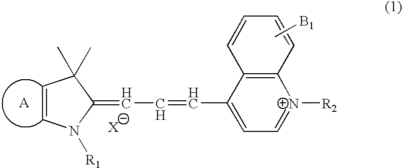

wherein A is an aromatic group or a polycyclic aromatic group; B₁ is a hydrogen atom, a hydroxyl group, an alkyloxy group, a halogen, a nitro group, a nitroso group, a substituted and unsubstituted amine group, a substituted or unsubstituted sulfamoyl group (—SO₂NH₂); R₁, R₂ are same or different, substituted or unsubstituted, straight chain or branched alkyl group, alkenyl group, aralkyl group, alkoxycarbonyl group, alkoxycarboxyl group, alkoxyl group, alkyl hydroxyl group, alkylamino group, alkylcarbamoyl group, alkylsulfamoyl group, alkylalkoxyl group, alkyl halide group, alkylsulfonyl group or alkylcarboxyl group; and X⁻ is anion.

### CROSS-REFERENCE TO RELATED APPLICATION

This application claims the priority benefit of Taiwan application serial no. 92113053, filed on May 14, 2003.

### BACKGROUND OF THE INVENTION

1. Field of the Invention

The present invention relates to an optical recording medium. More particularly, the present invention relates to a dye for an optical recording medium.

2. Description of Related Art

Through the development of internet and the advancement of computer power, a variety of information are being generated. The speed of data management by computer has expanded from the initial stage of number management to the management of a large amount of textual, sound, image data, and to the management of the current high quality moving picture. The information recording medium has developed from the early stage of magnetic recording, to hard disk recording and to the latest optical recording.

Optical recording can provides the following advantages over magnetic recording and semiconductor memory device: high storage capacity, small volume, stability over long storage period, low production cost, high compatibility and low error rate. The market for optical recording medium thus has increased drastically. In the various types of optical recording medium, the most popular one is the so-called Compact Disc-Recordable (CD-R), wherein the recording principle is based on an application of a laser beam at a wavelength of 770 nm to 830 nm for recording and assessing information.

Due to the constant demand on increasing the storage density and capacity of an optical recording medium, the original 650 MB capacity CD-R can no longer accommodate the audio-visual demand of the next generation. The industry, therefore, introduces the Digital Versatile Disc-Recordable (DVD-R) type of optical recording medium with a memory capacity several times of that of the CD-R. The Digital Versatile Disc-Recordable (DVD-R) has the capability for recording and retrieving information in a high density because semiconductor lasers having shorter wavelengths than that for the conventional CD-R (for example, lasers having wavelengths 600 nm to 680 nm) are used for recording and retrieving information. DVD-R is thereby going to be a major optical information recording medium in the future.

The write-once version of the DVD-R uses an organic dye as a recording layer. Recording is performed by irradiating a short-wavelength laser to the dye-based recording layer. Upon the absorption of the laser beam energy, the irradiated portion of the recording layer will undergo a thermal deformation. This basically forms a pit that represents the digital bit being recorded. The most commonly used organic dye includes cyanine dye, azo dye, benzofuryl ketone dye, indigo dye, etc. The above dyes can be used as a recording layer for an optical recording medium is simply because they can be spin-coated on a substrate. Compared to the vacuum evaporation coating method, the application of the spin coating method not only reduces the processing time, the production cost is also reduced. Thus, the stability of-the organic dyes and the solubility characteristics of the organic dyes in organic solvents are very important. To identify the type of organic dyes that can be applied as an optical recording medium is currently the most important research and development topic.

### SUMMARY OF THE INVENTION

Accordingly, the present invention provides an optical recording medium dye, wherein the dye has a broad absorption range and its thermal stability is favorable.

The present invention provides an optical recording medium dye, wherein this optical recording medium dye comprises the following structure (1) or structure (2)

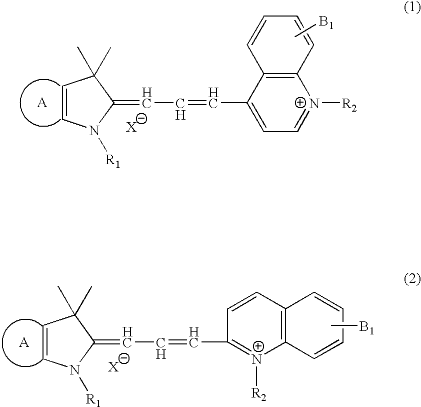

wherein A is an aromatic or a polycyclic aromatic group; B is a hydrogen atom, a hydroxyl group, an alkyloxy group, a halogen, a nitro group, a nitroso group, a substituted or unsubstituted amine group (—NH₂), a substituted or unsubstituted sulfamoyl group (SO₂NH₂). R₁ and R₂ are the same or different, substituted or unsubstituted, straight chain alkyl group, branched alkyl group, alkenyl group, aralkyl group, alkoxycarbonyl group, alkoxycarboxyl group, alkoxyl group, alkyl hydroxyl group, alkylamino group, alkylcarbamoyl group, alkylsulfamoyl group, alkalkoxyl group, alkyl halide group, alkylsulfonyl group or alkylcarboxyl group, and X⁻ is an anion.

A comprises the following structure (3), structure (4), structure (5):

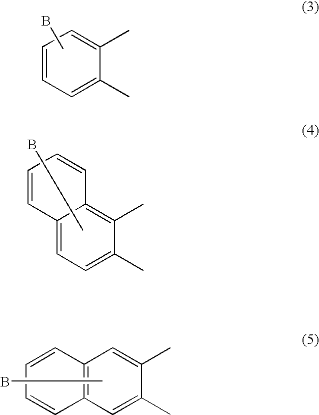

wherein B is a hydrogen atom, a hydroxyl group, an alkyloxy group, a halogen, a nitro group, a nitroso group, a substituted or an unsubstituted amine group (—NH₂), a substituted or an unsubstituted sulfamoyl group (—SO₂NH₂).

The present invention further provides an optical recording medium dye wherein its structure is different from that of a conventional cyanine dye. However, results of the spectroscopic analysis indicate that the absorption wavelength of the optical medium dye of the present invention is within the range of 500 nm to 650 nm. Further, the thermal characteristics and the solubility of the optical medium dye of the present invention are favorable. Therefore, the optical medium dye of the present invention is applicable as a recording layer of a high speed optical recording medium.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a diagram showing the maximum absorption wavelength of the chemical compound in Experiment 1.

FIG. 2 is a diagram showing the maximum absorption wavelength of the chemical compound in Experiment 2.

FIG. 3 is a diagram showing the maximum absorption wavelength of the chemical compound in Experiment 3.

FIG. 4 is a diagram showing the maximum absorption wavelength of the chemical compound in Experiment 4.

FIG. 5 is a diagram showing the maximum absorption wavelength of the chemical compound in Experiment 5.

FIG. 6 is a diagram showing the maximum absorption wavelength of the chemical compound in Experiment 6.

FIG. 7 is a diagram showing the maximum absorption wavelength of the chemical compound in Experiment 7.

FIG. 8 is a diagram showing the maximum absorption wavelength of the chemical compound in Experiment 8.

### DESCRIPTION OF THE EMBODIMENTS

The following is a detailed disclosure of an optical recording medium dye of the present invention.

The present invention provides an optical recording medium dye, wherein this optical recording medium dye comprises the following structure (1) or structure (2):

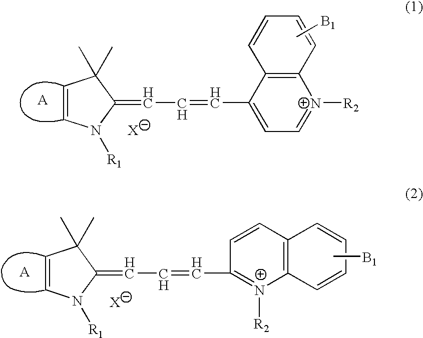

In the above structures (1) and (2), A includes an aromatic or a polycyclic aromatic compound having the following structure (3), structure (4) or structure (5).

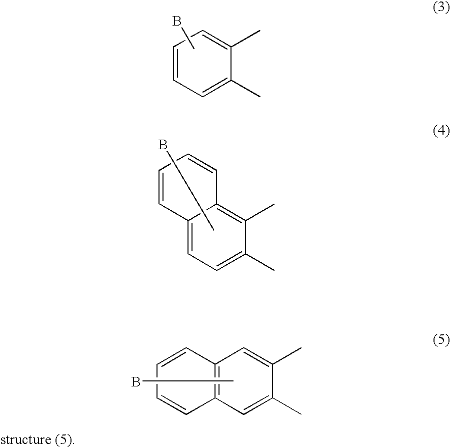

In the above structures (1) to (5), B and B₁ are, respectively, a hydrogen atom, a hydroxyl group, an alkyloxy group, a halogen, a nitro group, a nitroso group, a substituted or unsubstituted amine group (—NH₂), a substituted or unsubstituted sulfamoyl group (SO₂NH₂). Halogen is, for example, fluorine, chlorine, bromine and iodine. The substituted amine group (—NH₂) is substituted with, for example, a substituted (at least one hydrogen) or unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group or heterocyclic group, etc. The substituted sulfamoyl group is substituted with, for example, a substituted (at least one hydrogen) or unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group or heterocyclic group, etc.

R₁ and R₂ are the same or different, substituted or unsubstituted, straight chain alkyl group, branched alkyl group, alkenyl group, aralkyl group, alkoxycarbonyl group, alkoxycarboxyl group, alkoxyl group, alkyl hydroxyl group, alkylamino group, alkylcarbamoyl group, alkylsulfamoyl group, alkalkoxyl group, alkyl halide group, alkylsulfonyl group or alkylcarboxyl group.

The straight chain and branched alkyl group includes, for example, a methyl group, an ethyl group, a propyl group, an iso-propyl group, a butyl group, an iso-butyl group, a tert-butyl group, an 1-methyl butyl group, a 2-methyl butyl group, a 3-methylbutyl group, a pentyl group, an iso-pentyl group, a neopentyl group, a tert-pentyl group, an 1-methylpentyl group, a 2-methylpentyl group, a 5-methylpentyl group, a hexyl group, an iso-hexyl group, a heptyl group or an octyl group. The alkenyl group includes, for example, a vinyl group, an 1-propenyl group, a 2-propenyl group, an iso-propenyl group, a 2-butenyl group, an 1,3-butadienyl group and a 2-pentenyl group. The aralkyl group includes 5-methylene groups, typically 1 to 3 methylene groups, wherein two ends are connected to a monocyclic or polycyclic, saturated or unsaturated hydrocarbon group or heterocyclic group, for example, a phenyl group, a biphenyl group, an o-tolyl group, a m-tolyl group, a p-tolyl group, an o-cumenyl group, a m-cumenyl group, a p-cumenyl group, a xylyl group, a mesityl group, a styryl group, a cinnamoyl group, a naphthyl group. The alkoxycarbonyl group includes a methoxycarbonyl group, an ethoxycarbonyl group, a n-propoxycarbonyl group, an iso-propoxycarbonyl group, a n-butoxycarbonyl group, an iso-butoxycarbonyl group, a tert-butoxycarbonyl group, etc. The alkoxycarboxyl group includes a methoxycarboxyl group, an ethoxycarboxyl group, a n-propoxycarboxyl group, an iso-propoxycarboxyl, a n-butoxycarboxyl, an iso-butoxycarboxyl group, a tert-butoxycarboxyl, etc. The alkoxyl group includes a methoxyl group, an ethoxyl group, a n-propoxyl group, an isopropoxyl group, a n-butoxyl group, an i-butoxyl group, a t-butoxyl group, a pentoxyl group, etc. The alkyl hydroxyl group includes a methyoxyhydroxyl group, an ethoxyhydroxyl group, an n-propoxyhydroxyl group, an iso-propoxyhydroxyl group, a n-butoxyhydroxyl group, an iso-butoxyhydroxyl group, a tert-butoxyhydroxyl group, etc. The alkylamino group includes a methylamino group, an ethylamino group, a n-propylamino group, a n-butylamino group, a dimethylamino group, a diethylamino group, etc. The alkylcarbamoyl group includes a methylcarbamoyl group, an ethycarbamoyl group, an n-propylcarbamoyl group, an iso-propylcarbamoyl group, an n-butylcarbamoyl group, an iso-butylcarbamoyl group, a tert-butylcarbamoyl group, etc. The alkylsulfamoyl group includes a methylsulfamoyl group, an ethylsulfamoyl group, an n-propylsulfamoyl group, an iso-propylsulfamoyl group, a n-butylsulfamoyl group, an iso-butylsulfamoyl group, a tert-butylsulfamoyl group, etc. The alkylcarboxyl group includes a methylcarboxyl group, an ethylcarboxyl group, a n-propylcarboxyl group, an iso-propylcarboxyl group, a n-butylcarboxyl group, an iso-butylcarboxyl group, a tert-butycarboxyl group, etc.

X— is an anion, wherein X⁻ can be an anion of an inorganic or an organic acid. The anion of an inorganic acid is, for example, an anion of a fluoric acid, a chloric acid, a bromic acid, an iodic acid, a perchloric acid, a periodic acid, a phosphoric acid, a phosphoric acid hexafluoride, an antimony hexafluoride, a tin acid hexafluoride, a fluoroboric acid, etc. The anion of an organic acid is, for example, an anion of a thiocyanic acid, a benzenesulfonic acid, a p-toluenesulfonic acid, an alkylsulfonic acid, a benzenecarboxylic acid, an alkylcarboxylic acid, a trihaloalkylcarboxylic acid, a trihaloalkylsulfonic acid, a nicotinic acid or a thiocyanate (SCN^{-), etc.}

The synthesis of the optical recording medium dye of the present invention is accomplished by magnetically stirring the chemical compound of following structure (6), structure (7) or structure (8), the chemical compound of the following structure (9) or (10) in an organic solvent (for example, pyridine). Acetic anhydride is added to the above reaction mixture solution, followed by adding an organic salt solution or an inorganic salt solution. The resulting mixture solution is further reacted under heat. Deionized water is then added to bring the temperature of the reaction mixture solution to room temperature. Subsequent to filtering, washing, drying, the optical recording medium dye (as shown in the above structure (1) and structure (2)) is resulted.

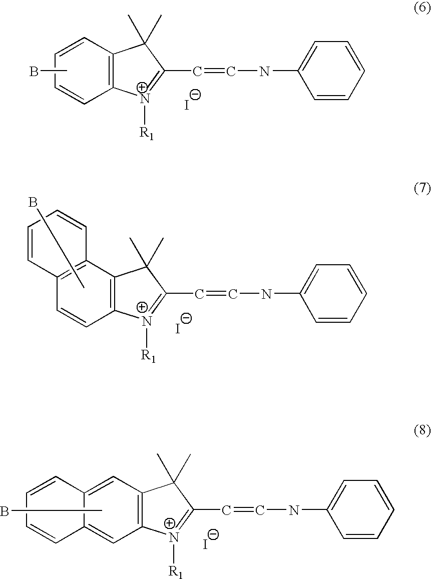

In the above structures (6) to (8), B is a hydrogen atom, a hydroxyl group, an alkyloxy group, a halogen group, a nitro group, a nitroso group, a substituted or unsubstituted amine group (—NH₂), a substituted or unsubstituted sulfamoyl group (SO₂NH₂). Halogen is, for example, fluorine, chlorine, bromine or iodine. The substituted amine group (—NH₂) is substituted with, for example, a substituted (at least one hydrogen) or unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group or heterocyclic group, etc. The substituted sulfamoyl group is substituted with, for example, a substituted (at least one hydrogen) or unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group or heterocyclic group, etc. R₁ is a substituted or unsubstituted, straight chain alkyl group, branched alkyl group, alkenyl group, aralkyl group, alkoxycarbonyl group, alkoxycarboxyl group, alkoxyl group, alkyl hydroxyl group, alkylamino group, alkylcarbamoyl group, alkylsulfamoyl group, alkalkoxyl group, alkyl halide group, alkylsulfonyl group or alkylcarboxyl group.

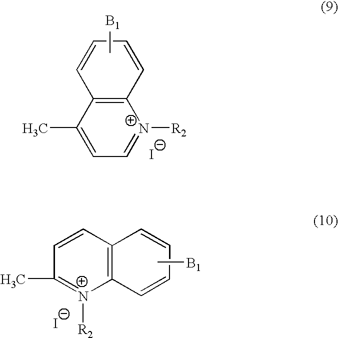

In the above structures (9) and (10), B₁ is a hydrogen atom, a hydroxyl group, an alkyloxy group, a halogen group, a nitro group, a nitroso group, an amine group (—NH₂) with one hydrogen being substituted or unsubstituted, a sulfamoyl group (SO₂NH₂) with one hydrogen being substituted or unsubstituted. Halogen is, for example, fluorine, chlorine, bromine and iodine. The substituted amine group (—NH₂) is substituted with a substituted (at least one hydrogen) or an unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group or heterocyclic group, etc. The substituted sulfamoyl group is substituted with, for example, a substituted (at least one hydrogen) or an unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group or heterocyclic group, etc. R₂ is a substituted or unsubstituted, straight chain alkyl group, branched alkyl group, alkenyl group, aralkyl group, alkoxycarbonyl group, alkoxycarboxyl group, alkoxyl group, alkyl hydroxyl group, alkylamino group, alkylcarbamoyl group, alkylsulfamoyl group, alkalkoxyl group, alkyl halide group, alkylsulfonyl group or alkylcarboxyl group.

To validate the optical property and the thermal property of the above optical recording medium dye, optical dyes of the present invention synthesized according the above fabrication method in experiment 1 to experiment 8 are discussed in detail below. The present invention is not limited to the dyes in experiment 1 to experiment 8.

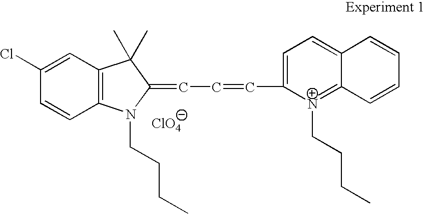

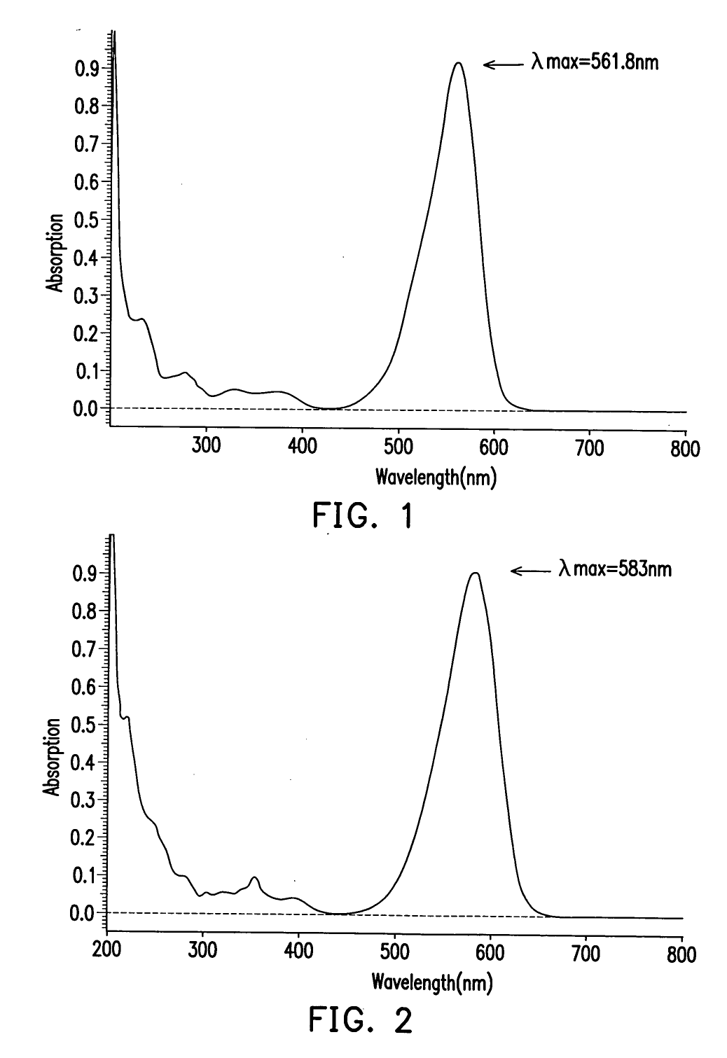

The UV-visible absorption spectrum of the chemical compound in experiment 1 is shown in FIG. 1. As shown in FIG. 1, the absorption wavelength of the chemical compound in experiment 1 is 561.8 nm. Further, the result of the thermal gravimetric analysis indicates that the sudden weight loss of the chemical compound in experiment 1 occurs at a temperature of about 280.2 degrees Celsius. Accordingly, the thermal breakdown temperature of the dye in experiment 1 can be as high as about 280.2 degrees Celsius.

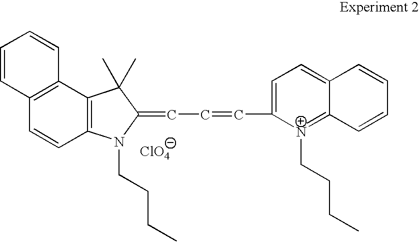

The UV-visible absorption spectrum of the chemical compound in experiment 2 is shown in FIG. 2. As shown in FIG. 2, the absorption wavelength of the chemical compound in experiment 2 is 583.0 nm. Further, the result of the thermal gravametric analysis indicates that the sudden weight loss of the chemical compound in experiment 2 occurs at a temperature of about 268.6 degrees Celsius. Accordingly, the thermal breakdown temperature of the dye in experiment 2 can be as high as about 268.6 degrees Celsius.

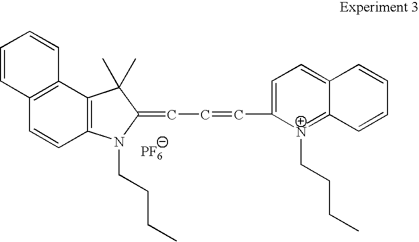

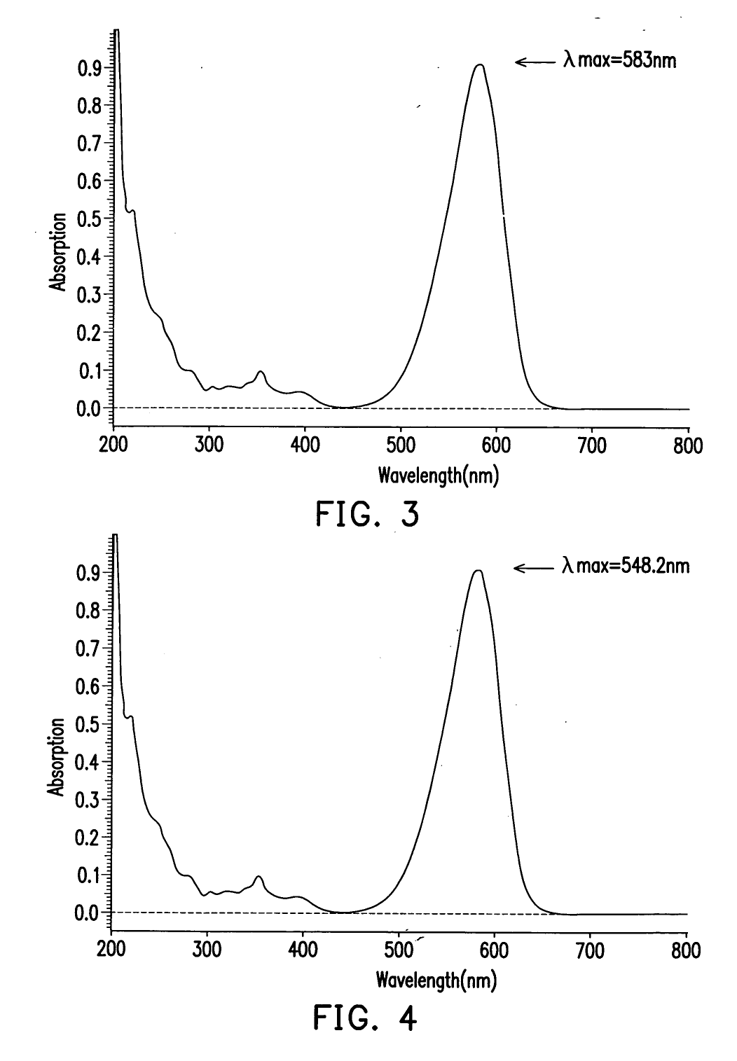

The UV-visible absorption spectrum of the chemical compound in experiment 3 is shown in FIG. 3. As shown in FIG. 3, the absorption wavelength of the chemical compound in experiment 3 is 583.0 nm. Further, the result of the thermal gravametric analysis indicates that the sudden weight loss of the chemical compound in experiment 3 occurs at a temperature of about 274.2 degrees Celsius. Accordingly, the thermal breakdown temperature of the dye in experiment 1 can be as high as about 274.2 degrees Celsius.

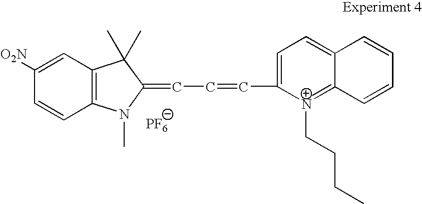

The UV-visible absorption spectrum of the chemical compound in experiment 4 is shown in FIG. 4. As shown in FIG. 4, the absorption wavelength of the chemical compound in experiment 4 is 548.2 nm. Further, the result of the thermal gravametric analysis indicates that the sudden weight loss of the chemical compound in experiment 4 occurs at a temperature of about 309.5 degrees Celsius. Accordingly, the thermal breakdown temperature of the dye in experiment 1 can be as high as about 309.5 degrees Celsius.

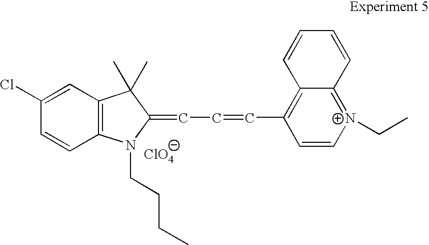

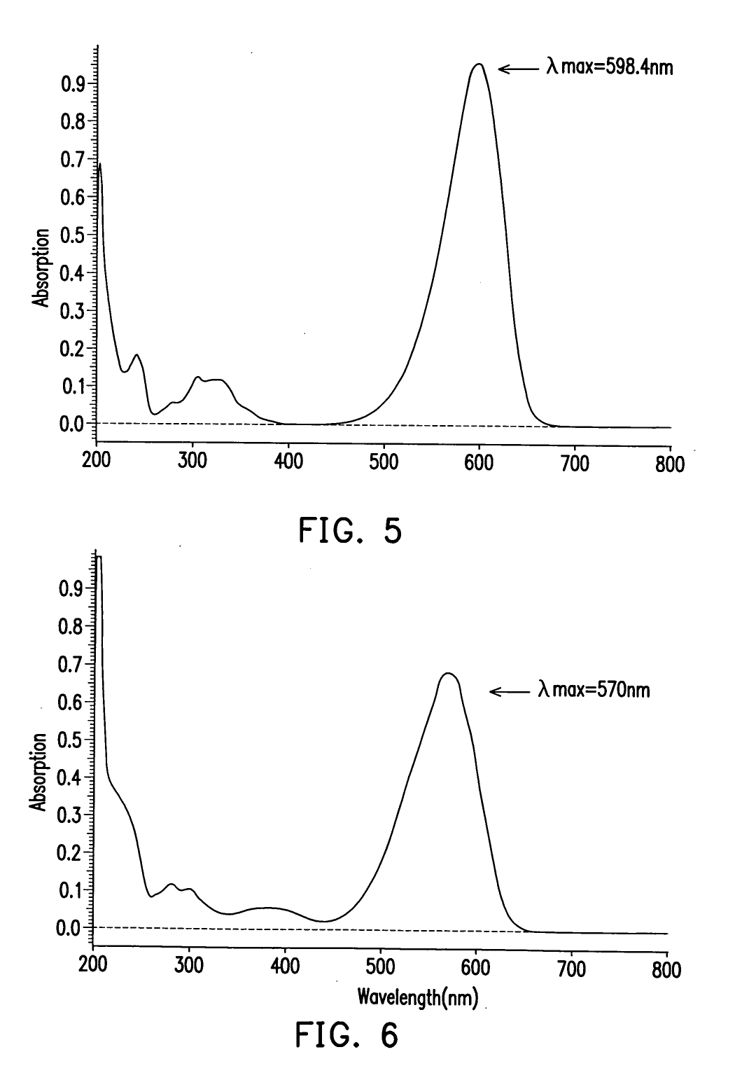

The UV-visible absorption spectrum of the chemical compound in experiment 5 is shown in FIG. 5. As shown in FIG. 5, the absorption wavelength of the chemical compound in experiment 5 is 598.4nm. Further, the result of the thermal gravametric analysis indicates that the sudden weight loss of the chemical compound in experiment 5 occurs at a temperature of about 266.3 degrees Celsius. Accordingly, the thermal breakdown temperature of the dye in experiment 5 can be as high as about 266.3 degrees Celsius.

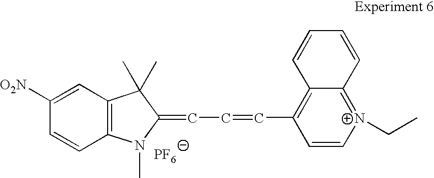

The UV-visible absorption spectrum of the chemical compound in experiment 6 is shown in FIG. 6. As shown in FIG. 6, the absorption wavelength of the chemical compound in experiment 6 is 570.0 nm. Further, the result of the thermal gravametric analysis indicates that the sudden weight loss of the chemical compound in experiment 6 occurs at a temperature of about 302.7 degrees Celsius. Accordingly, the thermal breakdown temperature of the dye in experiment 6 can be as high as about 302.7 degrees Celsius.

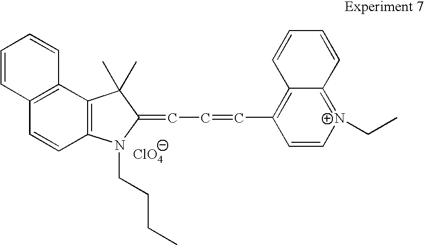

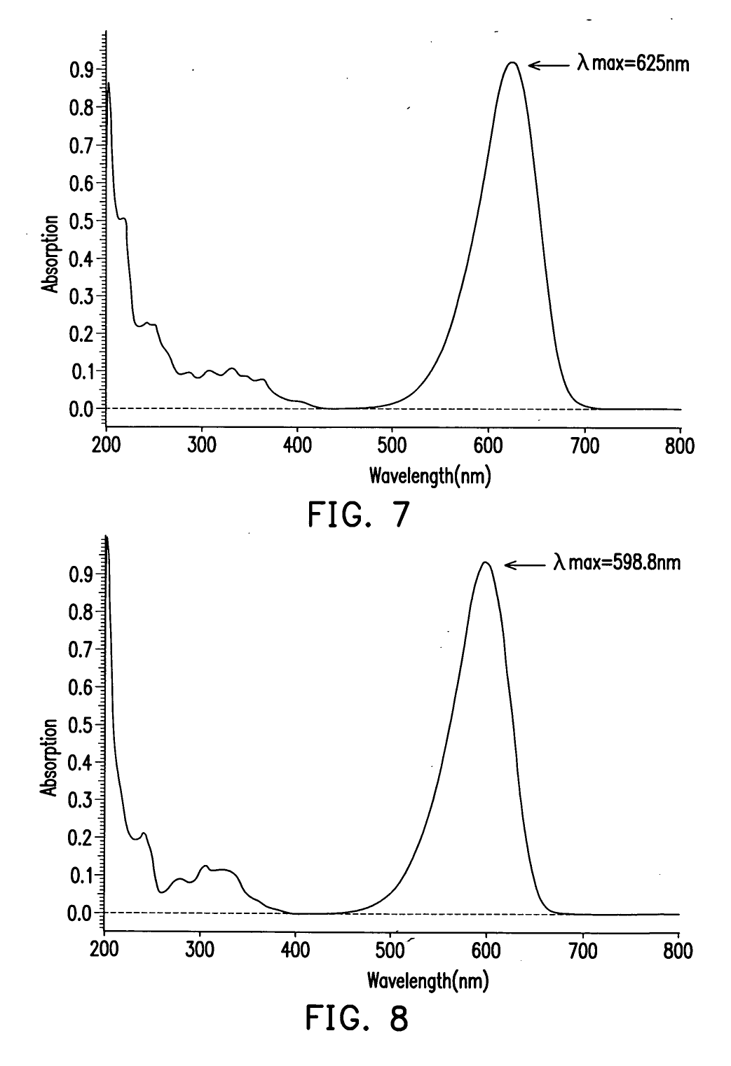

The UV-visible absorption spectrum of the chemical compound in experiment 7 is shown in FIG. 7. As shown in FIG. 7, the absorption wavelength of the chemical compound in experiment 7 is 625 nm. Further, the result of the thermal gravametric analysis indicates that the sudden weight loss of the chemical compound in experiment 7 occurs at a temperature of about 270.7 degrees Celsius. Accordingly, the thermal breakdown temperature of the dye in experiment 1 can be as high as about 270.7 degrees Celsius.

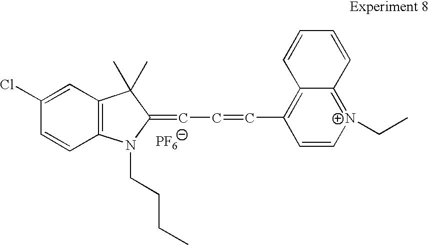

The UV-visible absorption spectrum of the chemical compound in experiment 8 is shown in FIG. 8. As shown in FIG. 8 the absorption wavelength of the chemical compound in experiment 8 is 598.8 nm. Further, the result of the thermal gravametric analysis indicates that the sudden weight loss of the chemical compound in experiment 8 occurs at a temperature of about 309.8 degrees Celsius. Accordingly, the thermal breakdown temperature of the dye in experiment 1 can be as high as about 309.8 degrees Celsius.

Detection of the Optical Thermal Characteristics

The results of the thermal gravametric analysis and the ultraviolent-visible absorption spectroscopy of the optical recording medium dyes obtained in the above experiment 1 to experiment 8 are summarized in Table 1. The thermal gravametric analysis and the ultraviolent-visible absorption spectroscopy detect, respectively, the thermal breakdown temperature (the temperature of a 5% thermal weight loss) and the optical property (the largest absorption wavelength, absorption coefficient of the solution). As shown in Table 1, the absorption wavelengths of the optical recording medium dyes of the present invention are within the range of 500 nm to 650 nm. Further, the thermal breakdown temperatures are above 250 degrees Celsius. Accordingly, the optical recording medium dyes of the present invention comprises a broad wavelength range and has favorable thermal stability and solubility.

TABLE 1

| | Maximum | Thermal | | |
|---|---|---|---|---|
| | Absorption | Breakdown | | Solubility in |
| | Wavelength | Temperature | Absorption | Tetrafluoro- |
| | (methanol/nm) | (° C.) | Coefficient | propanol |
| Experiment | 561.8 | 280.2 | 1.19 × 10⁵ | 10.10 |
| 1 | | | | |
| Experiment | 583.0 | 268.6 | 1.14 × 10⁵ | 6.31 |
| 2 | | | | |
| Experiment | 583.0 | 274.2 | 1.15 × 10⁵ | 0.70 |
| 3 | | | | |
| Experiment | 548.2 | 309.5 | 1.07 × 10⁵ | 0.54 |
| 4 | | | | |
| Experiment | 598.4 | 266.3 | 0.98 × 10⁵ | 19.47 |
| 5 | | | | |
| Experiment | 570.0 | 302.7 | 0.83 × 10⁵ | 1.43 |
| 6 | | | | |
| Experiment | 625.0 | 270.7 | 1.07 × 10⁵ | 11.33 |
| 7 | | | | |
| Experiment | 598.8 | 309.8 | 1.01 × 10⁵ | 2.12 |
| 8 | | | | |

Optical Recording Medium

The optical recording medium dyes obtained in the above experiment 1 to experiment 8 are each dissolved in 10 ml of tetrafluoropropanol and is then filtered using a filter paper with a pore diameter of 0.25 μm to obtain a filtrate. Using the spin-on coating method, 5 ml of the filtrate is spin coated on a substrate with a diameter of 12 cm at a speed of 500 rpm to form a coated layer on the substrate. The surface of the above substrate comprises a track having a depth of about 160 nm, and a width of about 0.3 μm. The aforementioned substrate coated with the optical dye is then dried under a temperature of 85° C. for 20 minutes. The aforementioned substrate is a transparent substrate with a signaled surface. The transparent substrate is formed with a material that includes, for example, polyester, polycarbonate (PC), polymethylmethacrylate (PMMA) or (metallocene catalyzed cyclo olefin copolymer).

Thereafter, the coated layer is sputtered with a thick metal layer (about 100 nm thick) as an anti-reflection layer. The anti-reflection layer is, for example, gold, silver, aluminum, copper, silver-titanium alloy, silver-chromium alloy and silver-copper alloy type of metal and other alloy material. Another substrate is then disposed on the anti-reflection layer, affixing to the substrate coated with the optical dye layer and sputtered with the anti-reflection layer, wherein the method for affixing the substrate to form a high density, recordable optical recording medium includes spin coating, printing, thermal-gluing, etc.

The above optical recording medium is span at a speed of 7 m/s and is irradiated with a semiconductor laser light with a power of 13 MW to record an eight to fourteen modulation (8-14 EFM). The wavelength of the semiconductor laser light is about 658 nm. A reproduction test is conducted to the above high density recordable optical recording medium using again the 685 nm semiconductor laser light to obtain a more favorable reproduction signal.

Accordingly, the absorption wavelength range (500 nm to 650 nm) for the optical recording medium dye is broader than the wavelength range (550 to 588) of the conventional cyanine dye. By adjusting the wavelength range, the optical recording medium dye of the present invention provides a broader application.

Further, the thermal breakdown temperature of the optical recording medium dye of the present invention is higher than that of the conventional cyanine dye. The optical recording medium dye of the present invention therefore does not easily generate crack/deformation at the region peripheral to the signal track due to the accumulation of heat to ensure the accuracy of the recorded information.

Further, the solubility of the optical recording medium dye of the present invention is desirable in which the ease for processing is enhanced.

Further, the optical recording medium dye of the present invention can be broadly applied to various types of optical recording media, such as, compact disk (CD), Digital Versatile Disc (DVD), mini disc (MD), CDV, digital audio tape (DAT), CD-ROM, DVD-ROM, etc.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An optical recording medium dye, wherein the optical recording medium dye has a structure of: 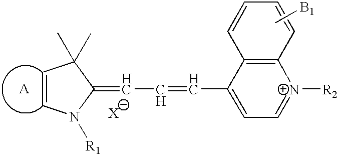
wherein A is a functional group selected from the group consisting of an aromatic compound and a polycyclic aromatic compound; B₁ is a functional group selected from the group consisting of a hydrogen atom, a hydroxyl group, an alkyloxy group, a halogen, a nitro group, a nitroso group, a substituted amine group (—NR_{2,} NHR), an unsubstituted amine group (—NH₂), a substituted sulfamoyl group (SO₂NR₂/SO₂NHR) and an unsubstituted sulfamoyl group (SO₂NH₂); R₁ and R₂ are functional groups selected from the group consisting of one of a same and a different, one of a substituted and an unsubstituted, straight chain alkyl group, branched alkyl group, alkenyl group, aralkyl group, alkoxycarbonyl group, alkoxycarboxyl group, alkoxyl group, alkyl hydroxyl group, alkylamino group, alkylcarbamoyl group, alkylsulfamoyl group, alkalkoxyl group, alkyl halide group, alkylsulfonyl group and alkylcarboxyl group; and X⁻ is an anion.

2. The optical recording medium dye of claim 1, wherein the functional group A in the structure has a structure (3), a structure (4) or a structure (5) 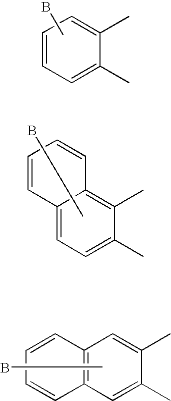
wherein B is a functional group selected from the group consisting of a hydrogen atom, a hydroxyl group, an alkyloxy group, a halogen, a nitro group, a nitroso group, a substituted amine group (—NHR/NR₂), an unsubstituted amine group (—NH₂), a substituted sulfamoyl group (—SO₂NHR/SO₂NR₂) and an unsubstituted sulfamoyl group (—SO₂NH₂).

3. The optical recording medium dye of claim 2, wherein the substituted amine group (—NHR/NR₂) is substituted with a functional group selected from the group consisting of one of a substituted and an unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group and heterocyclic group.

4. The optical recording medium dye of claim 2, wherein the substituted sulfamoyl group is substituted with a functional group selected from the group consisting of one of a substituted and an unsubstituted, straight chain alkyl group, branched chain alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group and heterocyclic group.

5. The optical recording medium dye of claim 1, wherein the halogen is selected from the group consisting of a fluoride, a chloride, a bromide and an iodide.

6. The optical recording medium dye of claim 1, wherein the substituted amine group (—NHR/NR₂) substituted with a functional group selected from the group consisting of one of a substituted and an unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group and heterocyclic group.

7. The optical recording medium dye of claim 1, wherein the substituted sulfamoyl group is substituted with a functional group selected from the group consisting of one of a substituted and an unsubstituted, straight chain alkyl group, branched chain alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group and heterocyclic group.

8. The optical recording medium dye of claim 1, wherein X⁻ is an anion of an acid selected from the group consisting of a fluoric acid, a chloric acid, a bromic acid, an iodic acid, a perchloric acid, a periodic acid, a phosphoric acid, a phosphoric acid hexafluoride, an antimony hexafluoride, a tin acid hexafluoride, a fluoroboric acid, a thiocyanic acid, a benzenesulfonic acid, a p-toluenesulfonic acid, an alkylsulfonic acid, a benzenecarboxylic acid, an alkylcarboxylic acid, a trihaloalkylcarboxylic acid, a trihaloalkylsulfonic acid, a nicotinic acid and a thiocyanate (SCN⁻).

9. An optical recording medium dye, the optical recording medium dye comprise a structure (2) 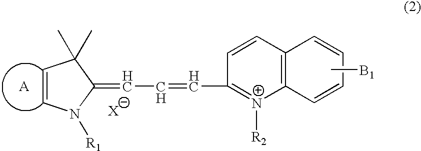
wherein A is a functional group selected from the group consisting of an aromatic compound and a polycyclic aromatic compound; B₁ is a functional group selected from the group consisting of a hydrogen atom, a hydroxyl group, an alkyloxy group, a halogen a nitro group, a nitroso group, a substituted amine group (—NHR/NR₂), an unsubstituted amine group (—NH₂), a substituted sulfamoyl group (SO₂NHR/SO₂NR₂) and an unsubstituted sulfamoyl group (SO₂NH₂); R₁ and R₂ are functional groups selected from the group consisting of one of a same and a different, one of a substituted and an unsubstituted, straight chain alkyl group, branched alkyl group, alkenyl group, aralkyl group, alkoxycarbonyl group, alkoxycarboxyl group, alkoxyl group, alkyl hydroxyl group, alkylamino group, alkylcarbamoyl group, alkylsulfamoyl group, alkalkoxyl group, alkyl halide group, alkylsulfonyl group and alkylcarboxyl group; and X⁻ is an anion.

10. The optical recording medium dye of claim 9, wherein the function group A in the structure (2) has a structure (3), a structure (4) or a structure (5) 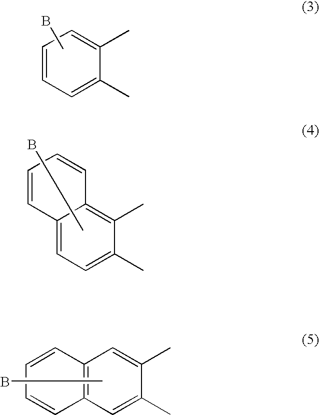
wherein B is a functional group selected from the group consisting of a hydrogen atom, a hydroxyl group, an alkyloxy group, a halogen, a nitro group, a nitroso group, a substituted amine group (—HR/NR₂), an unsubstituted amine group (—NH₂), a substituted sulfamoyl group (—SO₂NHR/SO₂NR₂) and an unsubstituted sulfamoyl group (—SO₂NH₂).

11. The optical recording medium dye of claim 10, wherein the substituted amine group (—NHR/NR₂) is substituted with a functional group selected from the group consisting of one of a substituted and an unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group and heterocyclic group.

12. The optical recording medium dye of claim 10, wherein the substituted sulfamoyl group is substituted with a functional group selected from the group consisting of one of a substituted and an unsubstituted, straight chain alkyl group, branched chain alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group and heterocyclic group.

13. The optical recording medium dye of claim 9, wherein the halogen is selected from the group consisting of a fluoride, a chloride, a bromide and an iodide.

14. The optical recording medium dye of claim 9, wherein the substituted amine group (—NHR/NR₂) is substituted with a functional group selected from the group consisting of one of a substituted and an unsubstituted, straight chain alkyl group, branched alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group and heterocyclic group.

15. The optical recording medium dye of claim 9, wherein the substituted sulfamoyl group is substituted with a functional group selected from the group consisting of one of a substituted and an unsubstituted, straight chain alkyl group, branched chain alkyl group, cycloalkyl group, alkoxy group, alkyl carbonyl group, straight chain alkenyl group, branched chain alkenyl group, cycloalkenyl group, hydroxyalkyl group, alkoxycarbonyl group, alkoxycarbonylallyl group, alkylthio group, alkylsulfonyl group, aryl group and heterocyclic group.

16. The optical recording medium dye of claim 9, wherein X⁻ is an anion of an acid selected from the group consisting of a fluoric acid, a chloric acid, a bromic acid, an iodic acid, a perchloric acid, a periodic acid, a phosphoric acid, a phosphoric acid hexafluoride, an antimony hexafluoride, a tin acid hexafluoride, a fluoroboric acid, a thiocyanic acid, a benzenesulfonic acid, a p-toluenesulfonic acid, an alkylsulfonic acid, a benzenecarboxylic acid, an alkylcarboxylic acid, a trihaloalkylcarboxylic acid, a trihaloalkylsulfonic acid, a nicotinic acid and a thiocyanate (SCN⁻).

17. An optical recording medium, comprising:
a first substrate, which is a transparent substrate comprising a signaled surface;
a recording layer, covering the first substrate, wherein the recording layer is formed comprising at least an optical recording medium dye as in one of claims 1 to 16; and
an anti-reflection layer, covering the optical recording layer.

18. The optical recording medium of claim 17, wherein the medium further comprises a second substrate, disposed on the anti-reflection layer.

19. The optical recording medium of claim 17, wherein a material for the anti-reflection layer is selected from the group consisting of gold, silver, aluminum, copper, silver-titanium alloy, silver-chromium alloy and silver-copper alloy type of metal and other alloy material.

20. The optical recording medium of claim 17, wherein a material for forming the first substrate and the second substrate is selected from the group consisting of polyester, polycarbonate (PC), polymethyimethacrylate (PMMA) and a metallocene catalyzed cyclo olefin copolymer.
